(19) United States
(12) Patent Application Publication
(10) Pub. No.: US 2026/0102148 A1
(43) Pub. Date: Apr. 16, 2026

(54) CLIP DEVICE AND CLIP UNIT

(71) Applicant: OLYMPUS MEDICAL SYSTEMS CORP,, Tokyo (JP)

(72) Inventor: Naoki TAKIZAWA, Musashino-shi (JP)

(21) Appl. No.: 19/418,502

(22) Filed: Dec. 12, 2025

Related U.S. Application Data
(63) Continuation of application No. PCT/JP2024/021745, filed on Jun. 14, 2024.
(60) Provisional application No. 63/508,598, filed on Jun. 16, 2023.

(51) Int. Cl. A61B 17/00

(52) U.S. Cl. CPC A61B 17/0057; A61B 17/00234; A61B 2017/00296; A61B 2017/00477; A61B 2017/00592; A61B 2017/00623; A61B 2017/00862

(57) ABSTRACT

A clip device includes a clip unit having an arm which opens and closes toward a front direction, a wire connected to the clip unit, and a sheath through which the wire is inserted and which extends in a longitudinal axis direction, in which the clip unit is separably connected to a distal end part of the sheath, and the clip unit is transitionable between a first configuration in which the front direction of the arm faces the same direction as the longitudinal axis direction and a second configuration in which the front direction of the arm faces a direction intersecting the longitudinal axis direction.

### BACKGROUND OF THE INVENTION

This application is a U.S. National Stage Filing under 35 U.S.C. §371 from International Application No. PCT/JP2024/021745, filed on June 14, 2024, and published as WO2024/257870 on December 19, 2024, which claims the benefit of priority to U.S. Provisional Application No. 63/508,598, filed on June 16, 2023; the benefit of priority of each of which is hereby claimed herein, and which applications and publication are hereby incorporated herein by reference in their entireties.

### FIELD OF THE INVENTION

The present invention relates to a clip device and a clip unit.

### DESCRIPTION OF RELATED ART

In endoscopic treatment, medical devices such as clip devices for treating living body tissue are used. These medical devices are introduced to a treatment site by an applicator (introducing device) that can be inserted through a channel of an endoscope.

Patent Document 1 describes a clipping device for treating living body tissue. In the clipping device described in Patent Document 1, a control wire for controlling a clip has a bent portion. When the bent portion of the control wire bends, the clip connected to the control wire moves greatly to come into contact with the tissue.

### SUMMARY OF THE INVENTION

A clip device according to a first aspect of the present invention includes a clip unit having an arm which opens and closes toward a front direction, a wire connected to the clip unit, and a sheath through which the wire is inserted and which extends in a longitudinal axis direction, in which the clip unit is separably connected to a distal end part of the sheath, and the clip unit is transitionable between a first configuration in which the front direction of the arm faces the same direction as the longitudinal axis direction and a second configuration in which the front direction of the arm faces a direction intersecting the longitudinal axis direction.

A clip device according to a second aspect of the present invention, which is a reloadable clip device, includes a clip unit having an arm which opens and closes toward a front direction, a wire connected to the clip unit, and a sheath through which the wire is inserted and which extends in a longitudinal axis direction, in which the clip unit is separably connected to a distal end part of the sheath, and the clip unit is transitionable between a first configuration in which the front direction of the arm faces the same direction as the longitudinal axis direction and a second configuration in which the front direction of the arm faces a direction intersecting the longitudinal axis direction.

A clip unit according to a third aspect of the present invention, which is a clip unit loaded into a sheath of an applicator, includes an arm which opens and closes toward a front direction, and a tubular member which is able to accommodate at least a part of the arm, and is able to transition between a first configuration in which the front direction of the arm faces the same direction as a longitudinal axis direction of the sheath, and a second configuration in which the front direction of the arm faces a direction intersecting the longitudinal axis direction.

### BRIEF DESCRIPTION OF THE DRAWINGS

FIG. 1 is a view illustrating a clip device according to a first embodiment.

FIG. 2 is a perspective view of a clip unit loaded into an applicator.

FIG. 3 is a perspective view of the clip unit loaded into the applicator.

FIG. 4 is a cross-sectional view of the clip unit.

FIG. 5 is a cross-sectional view of the clip unit.

FIG. 6 is a view illustrating transition of a shape of the clip unit.

FIG. 7 is a view illustrating transition of a shape of the clip unit.

FIG. 8 illustrates the clip unit that treats tissue by a tangential approach.

FIG. 9 is a view illustrating the clip unit in which a pair of arms have swung.

FIG. 10 is a view illustrating a separation step using the clip unit.

FIG. 11 is a view illustrating a modified example of a pressing tube of the clip unit.

FIG. 12 is a view illustrating another modified example of the pressing tube.

FIG. 13 is a view illustrating yet another modified example of the pressing tube.

FIG. 14 is a view illustrating a clip unit according to a second embodiment.

FIG. 15 is a view illustrating a tangential approach using the clip unit.

FIG. 16 is a view illustrating a tangential approach using the clip unit.

FIG. 17 is a view illustrating a clip unit according to a third embodiment.

FIG. 18 is a view illustrating a modified example of a curved member of the clip unit.

FIG. 19 is a view illustrating a clip unit according to a fourth embodiment.

### DETAILED DESCRIPTION OF THE INVENTION

### First embodiment

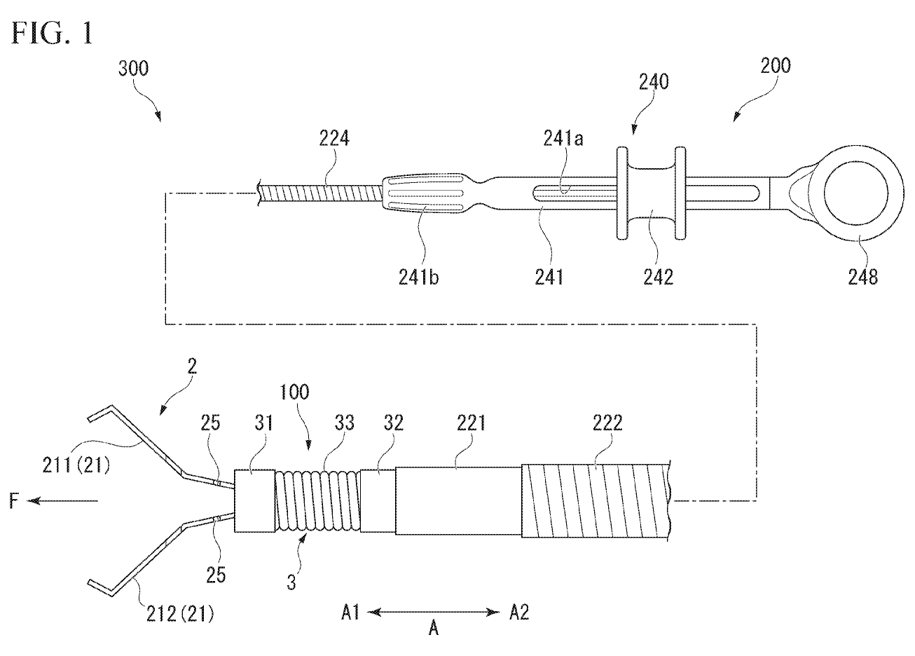

A first embodiment of the present invention will be described with reference to FIGS. 1 to 10.

FIG. 1 is a view illustrating a clip device 300 according to the present embodiment.

The clip device (clip system, endoscopic treatment tool) 300 includes a clip unit 100 and an applicator 200. The clip unit 100 is loaded into the applicator 200 that can be inserted through a channel of an endoscope and is introduced into a treatment site.

The clip device 300 may be configured such that the clip unit 100 can be reloaded into the applicator 200. In this case, a user can remove the used clip unit 100 from the applicator 200 and reload an unused clip unit 100 into the applicator 200.

In the following description, the clip unit 100 side in a longitudinal direction A of the clip device 300 will be referred to as a distal end side (distal side) A1 of the clip device 300, and a manipulation unit 240 side of the applicator 200 will be referred to as a proximal end side (proximal side) A2 of the clip device 300.

### Applicator 200

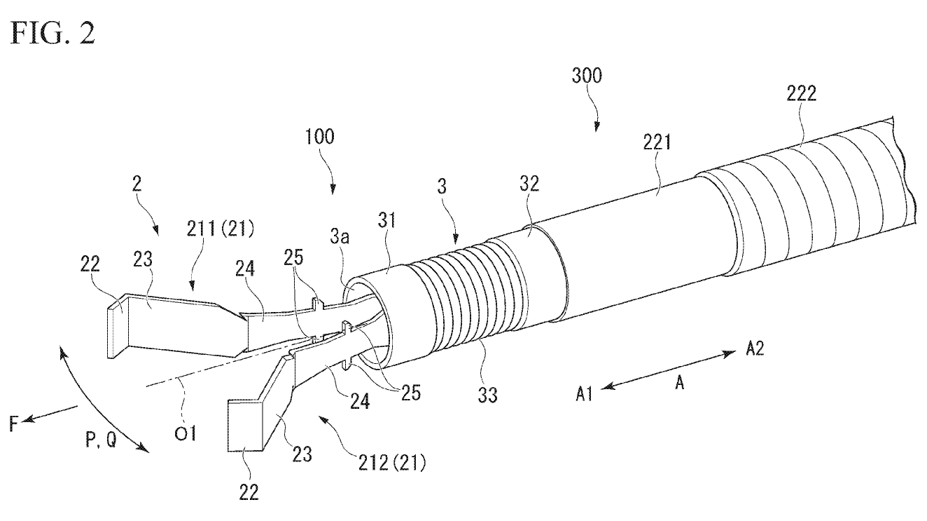

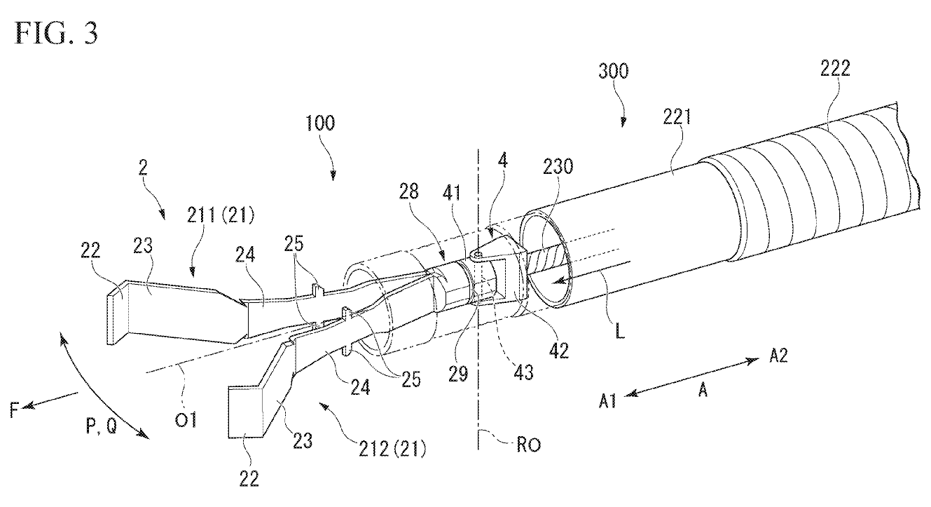

FIGS. 2 and 3 are perspective views of the clip unit 100 loaded into the applicator 200. The applicator (clip unit introducing device) 200 includes a sheath 220, a manipulation wire 230, and the manipulation unit 240. The applicator 200 is inserted through, for example, a treatment tool insertion channel of the endoscope to be used in combination with the endoscope. Therefore, the sheath 220 is formed to be sufficiently longer than a length of the treatment tool insertion channel of the endoscope. The sheath 220 has flexibility and bends in accordance with a curvature of an insertion portion of the endoscope.

The sheath 220 includes a distal end tip 221, a distal end-side coil 222, and a proximal-side coil 224, and is formed in an elongated tubular shape as a whole. The distal end-side coil 222 is disposed on a distal end portion side of the sheath 220. The distal end tip (distal end portion) 221 is formed in a cylindrical shape and is disposed at a distal end portion of the distal end-side coil 222.

The manipulation wire (power transmission member) 230 is inserted through the sheath 220 and attached to the clip unit 100 and the manipulation unit 240. As illustrated in FIG. 3, the manipulation wire 230 can protrude from the distal end tip (distal end portion) 221 toward the distal end side. The manipulation wire 230 advances and retracts the distal end tip (distal end portion) 221 along a longitudinal axis direction L of the sheath 220 to transmit power to a clip 2 of the clip unit 100.

The manipulation unit 240 includes a manipulation unit main body 241, a slider 242, and a thumb ring 248. The manipulation unit main body 241, the slider 242, and the thumb ring 248 are, for example, injection molded from a resin material. The manipulation unit main body 241 includes a slit portion 241a, and a rotation grip 241b on a distal end side. The slit portion 241a supports the slider 242 to be capable of advancing and retracting.

The slider 242 is attached to the manipulation unit main body 241 to be capable of advancing and retracting in the longitudinal axis direction, and a proximal end of the manipulation wire 230 is attached thereto. When the slider 242 advances and retracts along the manipulation unit main body 241, the manipulation wire 230 advances and retracts with respect to the sheath 220.

The thumb ring 248 is attached to a proximal end of the manipulation unit main body 241 to be rotatable around a longitudinal axis of the manipulation unit main body 241.

### Clip unit 100

The clip unit 100 is loaded into the distal end tip (distal end portion) 221 of the sheath 220 of the applicator 200. The clip unit 100 includes the clip 2, a pressing tube (tubular member) 3, and a curved member (connection member) 4.

The clip (clip arm) 2 includes a pair of arms 21 and a support portion 28 supporting the pair of arms 21. The support portion 28 is attached to a distal end of the curved member 4.

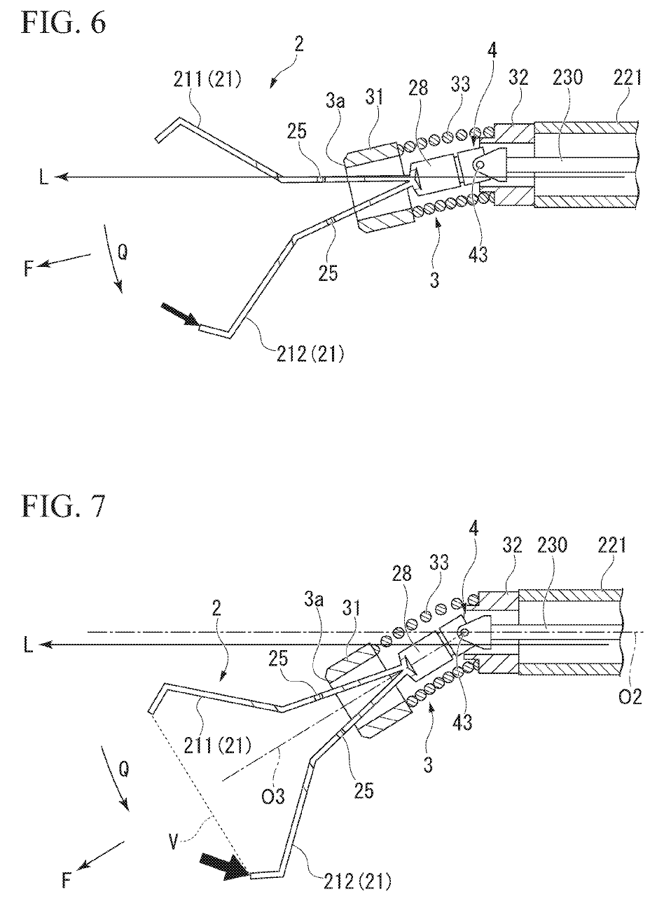

The pair of arms 21 can be opened and closed toward a front direction F and can transition between an open configuration (open state) and a closed configuration (closed state). The pair of arms 21 can swing in a swinging direction Q. The pair of arms 21 changes the front direction F by swinging (see FIGS. 6 and 7). As illustrated in FIGS. 2 and 3, a position of the pair of arms 21 in which the front direction F substantially coincides with the longitudinal axis direction L of the sheath 220 is referred to as an “initial position” of the pair of arms 21.

The pair of arms 21 includes a first arm 211 and a second arm 212. As illustrated in FIGS. 2 and 3, when the first arm 211 and the second arm 212 are disposed in the “initial position” without swinging, they are disposed on both sides with a central axis O1 in the longitudinal direction A interposed therebetween. Further, the clip 2 may have three or more arms.

The first arm 211 and the second arm 212 each have a claw 22, a gripping portion 23, a sliding portion 24, and an engagement portion 25 from the distal end side A1 to the proximal end side A2.

The claw 22 is formed by bending a distal end of each of the first arm 211 and the second arm 212 inward. The gripping portion 23 is formed in a flat plate shape and is a portion that grips tissue. The sliding portion 24 is a portion that elastically deforms when the pair of arms 21 are drawn into the pressing tube 3.

The engagement portion 25 is a portion that can engage with an inner circumferential surface of the pressing tube 3. The engagement portion 25 is a protruding portion that protrudes from the sliding portion 24. Specifically, the engagement portion 25 protrudes on both sides in a vertical direction perpendicular to the longitudinal direction A and an opening/closing direction P of the pair of arms 21.

The engagement portion 25 and the inner circumferential surface of the pressing tube 3 constitute a locking mechanism that locks the clip 2 in the closed configuration, as will be described later. When the pair of arms 21 are locked in the closed configuration by the locking mechanism, the pair of arms 21 cannot return to the open configuration. Further, the locking mechanism is not limited to the combination of the engagement portion 25 of the clip 2 and the inner circumferential surface of the pressing tube 3, but may have other configurations.

The support portion 28 supports the pair of arms 21 (the first arm 211 and the second arm 212) to be openable and closable in an opening/closing direction P. The pair of arms 21 supported by the support portion 28 are biased to be in the open configuration. Therefore, the pair of arms 21 of the clip 2 have a self-expanding force in the opening/closing direction P.

The support portion 28 has a separation portion 29 that is separably connected to a distal end member 41 of the curved member 4 on the proximal end side A2. For example, the separation portion 29 may be a breaking portion that breaks when a predetermined breaking force or more is applied by pulling. Also, the separation portion 29 may also be a deformable portion that is formed in a hook shape or the like and separates from the distal end member 41 due to plastic deformation.

The pressing tube (tubular member) 3 is a tubular member capable of accommodating at least a part of the clip 2. The pressing tube 3 has an internal space 38 through which the clip 2 advances and retracts in the longitudinal direction A. The pressing tube 3 can fix the clip 2, which has been drawn into the internal space 38, in the closed state.

The pressing tube (tubular member) 3 includes a distal end tube portion 31, a proximal end tube portion 32, and a flexible tube portion 33 connecting the distal end tube portion 31 and the proximal end tube portion 32.

The distal end tube portion 31 is a hard member formed of a metal or the like and is formed in an annular shape. The distal end tube portion 31 is provided at a distal end of the flexible tube portion 33. A distal end opening 3a is formed on the distal end side A1 of the distal end tube portion 31.

The proximal end tube portion 32 is a hard member formed of a metal or the like, and is formed in an annular shape. The proximal end tube portion 32 is provided at a proximal end of the flexible tube portion 33.

The flexible tube portion 33 has higher flexibility than the distal end tube portion 31 and the proximal end tube portion 32, and is bendable. In the present embodiment, the flexible tube portion 33 is a coil sheath. The flexible tube portion 33 has elasticity to return to a linear configuration in a natural state without being subjected to an external force.

The flexible tube portion 33 may be a tubular member formed in a cylindrical shape and having flexibility, such as a spring, a laser-cut pipe, or a flexible tube. These tubular members also have elasticity to return to a linear configuration in a natural state without being subjected to an external force.

The flexible tube portion 33 may be a tubular member to which cylindrical bending pieces formed of a hard material such as a metal are connected. Even if a single bending piece does not have flexibility, it is sufficient that a tubular member in which a plurality of bending pieces are connected has flexibility. Further, the number of bending pieces to be connected may be two.

When the distal end tube portion 31 and proximal end tube portion 32, and the flexible tube portion 33 are formed using different materials, one of the materials may be a material having lower magnetism than the other material. An influence of the clip unit 100 on MRI can be reduced.

The proximal end tube portion 32 of the pressing tube 3 is separably connected to the distal end tip 221 of the applicator 200. The proximal end tube portion 32 may be separably connected to the distal end tip 221 by, for example, an adhesive material. Also, the proximal end tube portion 32 may be separably connected to the distal end tip 221 by, for example, fitting. Also, the proximal end tube portion 32 may merely be in contact with the distal end tip 221.

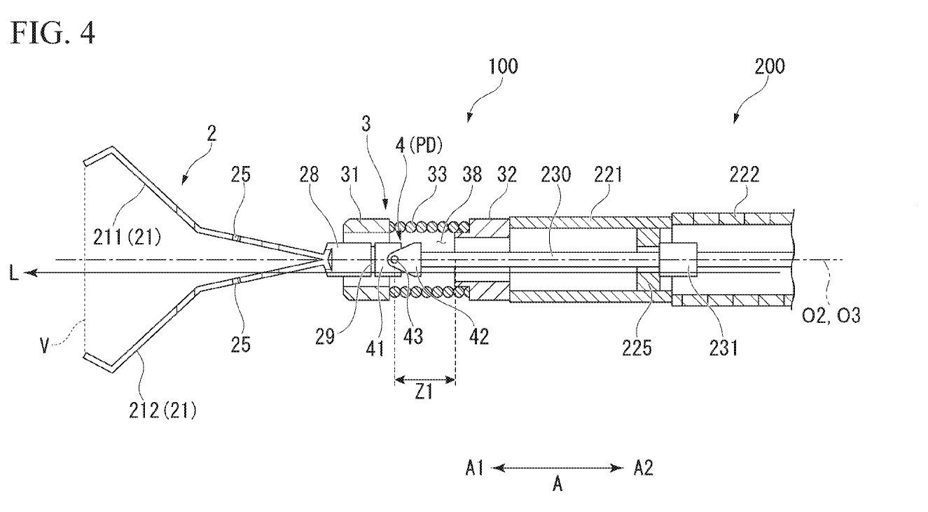

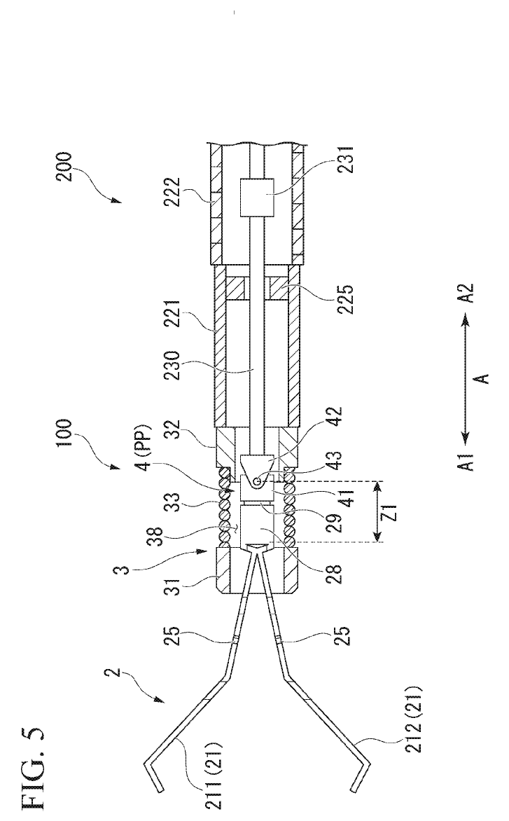

FIGS. 4 and 5 are cross-sectional views of the clip unit 100.

The curved member (connection member) 4 supports the pair of arms 21 to be swingable (rotatable) about a rotation axis RO extending in a direction substantially perpendicular to the longitudinal direction A. In the present embodiment, the swinging direction (rotating direction) Q of the pair of arms 21 about the rotation axis RO as a rotation center substantially coincides with the opening/closing direction P of the pair of arms 21. That is, a plane in which the pair of arms 21 swing (a plane through which the swinging pair of arms 21 pass) substantially coincides with a plane in which the pair of arms 21 open and close (a plane through which the opening/closing pair of arms 21 pass).

The curved member (connection member) 4 causes the pair of arms 21 to swing when a force equal to or greater than a predetermined value is applied to at least one of the first arm 211 and the second arm 212, such as when at least one of the first arm 211 and the second arm 212 comes into contact with living body tissue or when an external force equal to or greater than a predetermined value is applied to at least one of the first arm 211 and the second arm 212. In other cases, the support portion 28 does not allow the pair of arms 21 to swing.

The curved member (connection member) 4 includes the distal end member 41, a proximal end member 42, and a hinge (rotating portion) 43 that rotatably connects the distal end member 41 and the proximal end member 42 together.

The hinge (rotating portion) 43 connects the distal end member 41 and the proximal end member 42 so that the distal end member 41 rotates in the swinging direction (rotating direction) Q with respect to the proximal end member 42.

A position at which the curved member 4 can be disposed on the distal end side A1 is restricted to a distal end position PD (see FIG. 4). For example, when an enlarged diameter portion 231 provided on the manipulation wire 230 engages with a reduced diameter portion 225 provided in an internal space of the distal end tip 221 or the like, the curved member 4 cannot move further to the distal end side A1 than the distal end position PD.

When the pair of arms 21 are not locked in the closed configuration by the locking mechanism, a position at which the curved member 4 can be disposed on the proximal end side A2 is restricted to a proximal end position PP (see FIG. 5). The pair of arms 21 have a self-expanding force in the opening/closing direction. Therefore, even when the curved member 4 is pulled to the proximal end side A2 beyond the proximal end position PP, if a pulling force is released in a state in which the pair of arms 21 are not locked, the pair of arms 21 return to the open state while moving to the distal end side A1 due to the self-expanding force of the pair of arms 21. As a result, the curved member 4 advances to the proximal end position PP and is disposed at the proximal end position PP.

When the curved member 4 moves within a range from the distal end position PD to the proximal end position PP, it is desirable that the hinge 43 of the curved member 4 be disposed in the internal space 38 of the flexible tube portion 33 of the pressing tube 3. In this case, as illustrated in FIGS. 4 and 5, a movement range Z1 of the hinge 43 when the curved member 4 moves within the range from the distal end position PD to the proximal end position PP is included in the internal space 38 of the flexible tube portion 33. When the pair of arms 21 swing with respect to the curved member 4, since the flexible tube portion 33 bends in conjunction therewith, a swinging motion of the curved member 4 is not hindered by the pressing tube 3.

FIGS. 6 and 7 are views illustrating transitions of the shape of the clip unit 100.

The clip unit 100 can be transitioned between a linear configuration (first configuration) illustrated in FIG. 5 and a swinging configuration (second configuration) illustrated in FIG. 7.

The linear configuration (first configuration) is a configuration of the clip unit 100 in which the front direction F of the pair of arms 21 faces the same direction as the longitudinal axis direction L of the sheath 220 as illustrated in FIGS. 2 and 3. That is, as illustrated in FIG. 4, the linear configuration is a configuration in which a central axis O3 of the distal end tube portion 31 extends in the longitudinal axis direction L of the sheath 220. Also, the linear configuration is a configuration in which a virtual line segment V intersects a central axis O2 of the sheath 220 in the longitudinal axis direction L. Here, the virtual line segment V is a line segment connecting distal ends of the pair of arms 21 in the open configuration. Also, the linear configuration is a configuration in which a distal end of the first arm 211 and a distal end of the second arm 212 are disposed with the central axis O2 of the sheath 220 interposed therebetween. Also, the linear configuration is a configuration in which, in the pair of arms 21 in the open configuration, a distance between the distal end of the first arm 211 and the central axis O2 of the sheath 220 is substantially equal to a distance between the distal end of the second arm 212 and the central axis O2 of the sheath 220.

The swinging configuration (second configuration) is a configuration of the clip unit 100 in which the front direction F of the pair of arms 21 faces a direction intersecting the longitudinal axis direction L of the sheath 220 as illustrated in FIGS. 6 and 7. That is, as illustrated in FIG. 7, the swinging configuration is a configuration in which the central axis O3 of the distal end tube portion 31 faces a direction intersecting the longitudinal axis direction L of the sheath 220. Also, the swinging configuration is a configuration in which the virtual line segment V does not intersect the central axis O2 of the sheath 220. Also, the swinging configuration is a configuration in which the distal end of the first arm 211 and the distal end of the second arm 212 are disposed without sandwiching the central axis O2 of the sheath 220 therebetween. Also, the swinging configuration is a configuration in which, in the pair of arms 21 in the open configuration, the distance between the distal end of the first arm 211 and the central axis O2 of the sheath 220 is smaller or larger than the distance between the distal end of the second arm 212 and the central axis O2 of the sheath 220.

When a force equal to or greater than a predetermined value is applied to at least one of the first arm 211 and the second arm 212, the clip unit 100 swings in the swinging direction Q and transitions to the swinging configuration (second configuration). Specifically, the pair of arms 21 swing in the swinging direction Q, and the pressing tube 3 in contact with the pair of arms 21 bends.

The clip unit 100 can also transition from the swinging configuration (second configuration) to the linear configuration (first configuration). If the flexible tube portion 33 has sufficient elasticity, the clip unit 100 in the swinging configuration returns to the linear configuration in a natural state without being subjected to an external force.

### Operation and function of clip unit 100

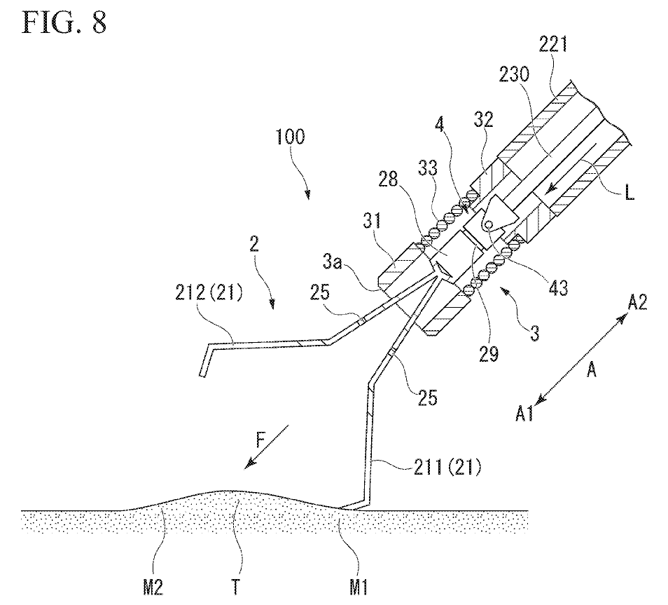

Next, an operation and function of the clip unit 100 will be described with reference to FIGS. 8 to 10.

### Approach step

The operator introduces the clip unit 100 loaded in the applicator 200 into the body via a channel of the endoscope. The operator moves the endoscope and the sheath

220 to bring the clip 2 of the clip unit 100 closer to a tissue T, which is a treatment region.

Methods for approaching the treatment region with the clip unit 100 include at least a frontal approach and a tangential approach. In the frontal approach, the operator disposes the clip unit 100 such that the longitudinal direction A of the clip unit 100 is substantially perpendicular to a surface of the treatment region by manipulating the endoscope and the sheath 220. In the tangential approach, the operator disposes the clip unit 100 such that the longitudinal direction A of the clip unit 100 extends along a surface of the treatment region by manipulating the endoscope and the sheath 220.

### Tangential approach

FIG. 8 illustrates the clip unit 100 treating the tissue T by the tangential approach. The operator brings the clip unit 100 in the linear configuration closer to the tissue T, which is a treatment region, by the tangential approach. The operator hooks the claw 22 of the first arm 211 onto a mucosa (first mucosa M1 in FIG. 8) that is on the endoscope side (near side) among the first mucosa M1 and a second mucosa M2 that are on both sides of a center of the tissue T.

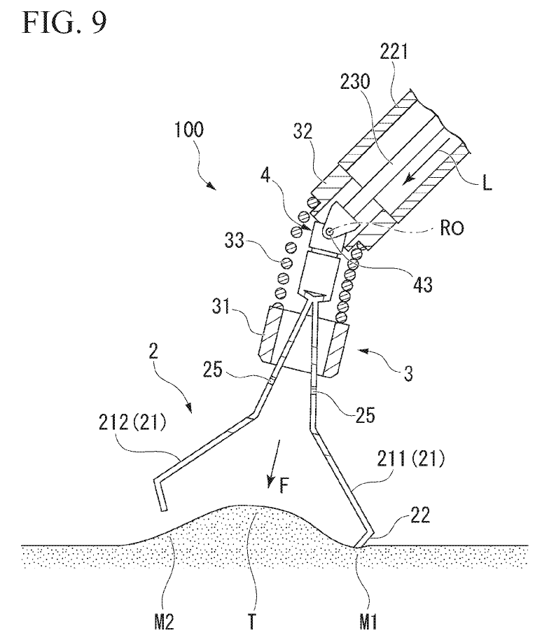

FIG. 9 is a view illustrating the clip unit 100 in which the pair of arms 21 have swung. The operator further brings the clip unit 100, in which the claw 22 is hooked on the first mucosa M1, closer to the tissue T, which is a treatment region. Specifically, the operator advances the applicator 200 with respect to the channel of the endoscope. Alternatively, the operator may further press the clip unit 100 against the tissue T by advancing the endoscope. As a result, as illustrated in FIG. 9, the pair of arms 21 swing around the rotation axis RO, and the clip unit 100 assumes the swinging configuration. When the second arm 212 comes into contact with the second mucosa M2, the pair of arms 21 are disposed so that the front direction F faces the front of the tissue T, which is a treatment region. The first mucosa M1 and the second mucosa M2 are grasped by the pair of arms 21.

### Pulling step

The user retracts the manipulation wire 230 by retracting the slider 242 along the manipulation unit main body 241. The curved member 4 connected to the manipulation wire 230 pulls the clip 2. When the clip 2 is pulled to the proximal end side A2, the pair of arms 21 are drawn into the pressing tube 3, and the pair of arms 21 engaged with the distal end opening 3a gradually close. When a pulling force of the manipulation wire 230 is released in this state, the clip 2 returns to the open state while moving to the distal end side A1 due to the self-expanding force of the pair of arms 21. The user can return the pair of arms 21 to the open state and re-grasp the tissue T (reopening).

The pressing tube 3 has the hard distal end tube portion 31 on the distal end side A1. Therefore, the distal end tube portion 31 and the pair of arms 21 have a high sliding ability, and the pair of arms 21 can smoothly advance and retract. Also, the pressing tube 3 also has a hard proximal end tube portion 32 on the proximal end side A2. Since the proximal end tube portion 32 is firmly in contact with the distal end tip 221 of the sheath 220, power of the manipulation wire 230 is easily transmitted to the clip unit 100.

### Lock step

The operator retracts the manipulation wire 230 by retracting the slider 242 along the manipulation unit main body 241. When the clip 2 is drawn into a predetermined position in the pressing tube 3, the engagement portion 25 serving as a locking mechanism engages with the inner circumferential surface of the pressing tube 3, and the pair of arms 21 are locked in the closed state. When the pair of arms 21 are locked in the closed state, the pair of arms 21 cannot return to the open state.

### Separation step

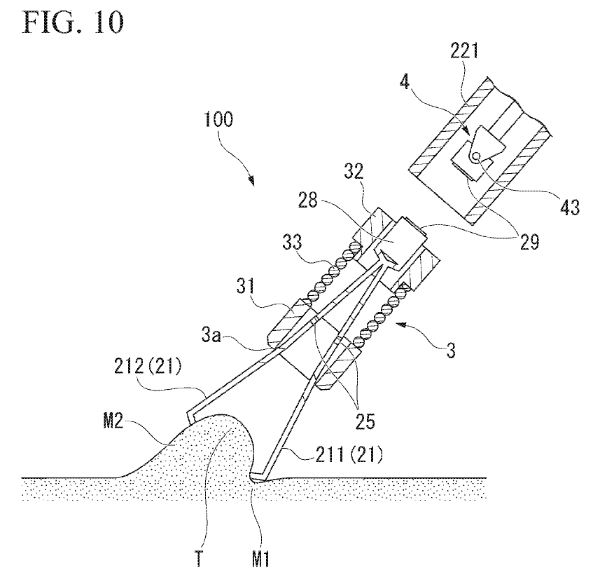

FIG. 10 is a view illustrating a separation step using the clip unit 100.

The operator further pulls the clip 2. A breaking force by pulling is applied to the separation portion 29 of the clip 2, and the breaking portion breaks. As a result, the clip 2 and the curved member 4 are separated. The operator retracts the sheath 220 and leaves the clip 2 in a state of ligating the first mucosa M1 and the second mucosa M2, together with the pressing tube 3, in the body.

According to the clip unit 100 of the present embodiment, the tissue T can be suitably grasped in a lumen. Even when the tissue T is treated by a tangential approach, the pair of arms 21 can be disposed to face the front of the tissue T, which is a treatment region, by swinging the entire clip unit 100. Since the entire clip unit 100 swings, the clip unit 100 can be easily disposed to face the front of the tissue T even in a narrow lumen.

While the first embodiment of the present invention has been described in detail as above with reference to the drawings, the specific configurations are not limited to the embodiment and may include design changes or the like within a range not departing from the gist of the present invention. Also, the components illustrated in the above-described embodiment and modified examples can be configured by appropriately combining them.

### Modified example 1

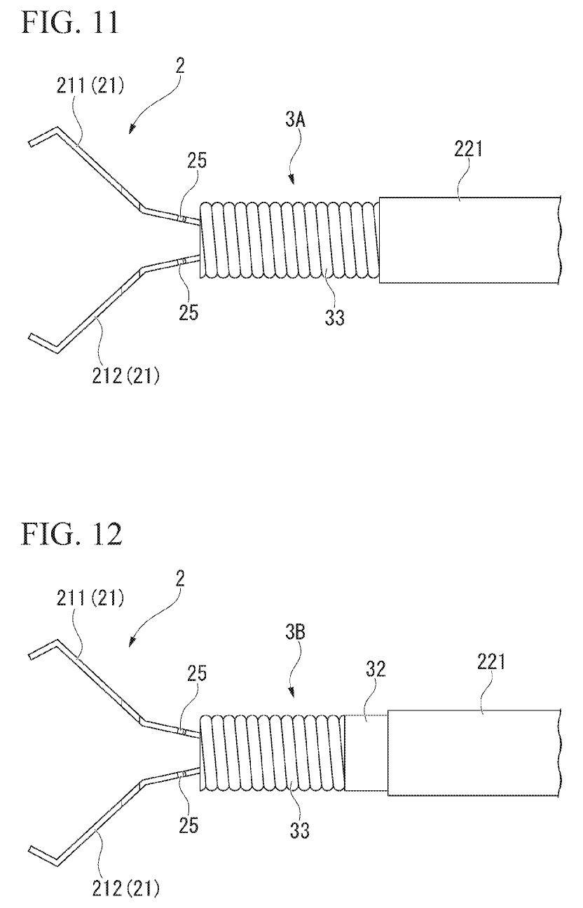

FIG. 11 is a view illustrating a pressing tube 3A which is a modified example of the pressing tube 3.

The pressing tube 3A has the flexible tube portion 33 and does not have the distal end tube portion 31 and the proximal end tube portion 32. The pressing tube 3A is constituted only by the flexible tube portion 33, and has lower manufacturing costs compared to the pressing tube 3.

### Modified example 2

FIG. 12 is a view illustrating a pressing tube 3B which is a modified example of the pressing tube 3.

The pressing tube 3B has the proximal end tube portion 32 and the flexible tube portion 33, but does not have the distal end tube portion 31. Since the proximal end tube portion 32 of the pressing tube 3B is firmly in contact with the distal end tip 221 of the sheath 220, power of the manipulation wire 230 is easily transmitted to the clip unit 100. The pressing tube 3B is constituted only by the proximal end tube portion 32 and the flexible tube portion 33, and has lower manufacturing costs compared to the pressing tube 3.

### Modified example 3

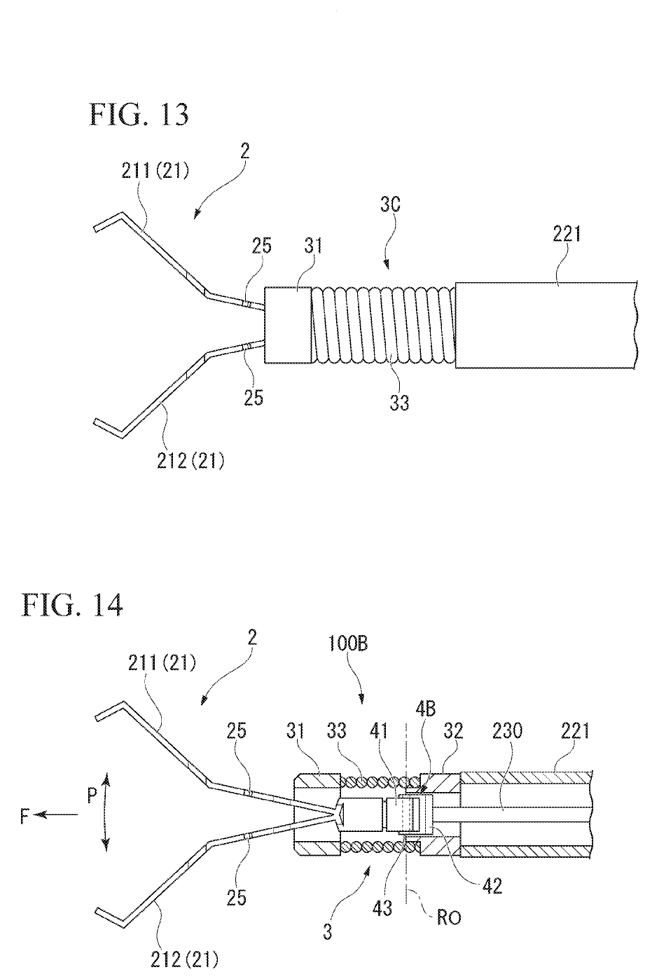

FIG. 13 is a view illustrating a pressing tube 3C which is a modified example of the pressing tube 3.

The pressing tube 3C has the distal end tube portion 31 and the flexible tube portion 33, but does not have the proximal end tube portion 32. The distal end tube portion 31 and the pair of arms 21 have a high sliding ability, and the pair of arms 21 can smoothly advance and retract. The pressing tube 3C is constituted only by the distal end tube portion 31 and the flexible tube portion 33, and has lower manufacturing costs compared to the pressing tube 3.

### Second embodiment

A second embodiment of the present invention will be described with reference to FIGS. 14 to 16. In the following description, components that are common to those already described will be denoted by the same reference signs and duplicate description will be omitted.

FIG. 14 is a view illustrating a clip unit 100B according to the present embodiment.

The clip unit 100B is loaded into a distal end tip (distal end portion) 221 of a sheath 220 of an applicator 200, similarly to the clip unit 100 of the first embodiment.

The clip unit 100B includes a clip 2, a pressing tube (tubular member) 3, and a curved member (connection member) 4B.

The curved member (connection member) 4B supports a pair of arms 21 to be swingable (rotatable) about a rotation axis RO extending in a direction substantially perpendicular to a longitudinal direction A. In the present embodiment, a swinging direction (rotating direction) Q of the pair of arms 21 about the rotation axis RO as a rotation center is substantially orthogonal to an opening/closing direction P of the pair of arms 21.

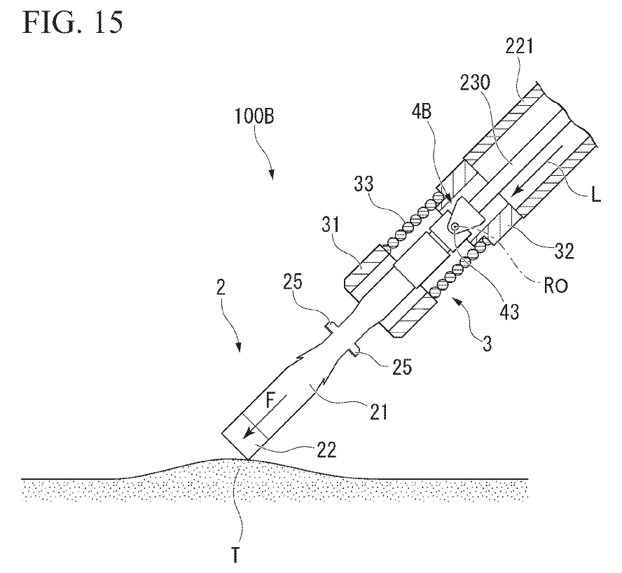

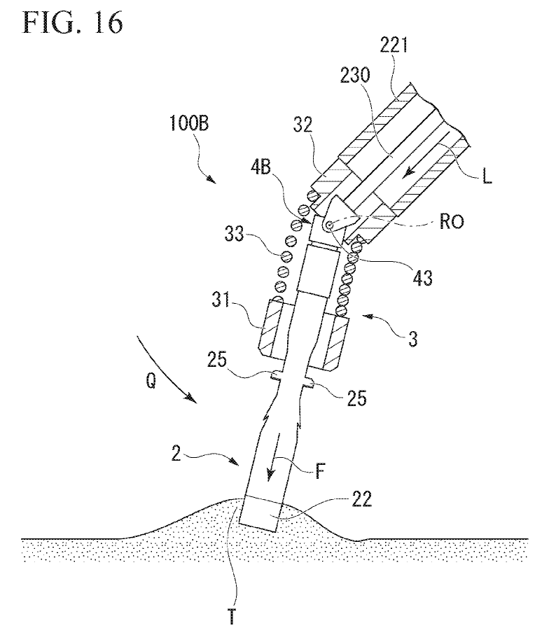

FIGS. 15 and 16 are views illustrating a tangential approach using the clip unit 100B. An operator further brings the clip unit 100B, in which two claws 22 are hooked on a mucosa, closer to a tissue T. Specifically, the operator advances the applicator 200 with respect to a channel of the endoscope. Alternatively, the operator may further press the clip unit 100B against the tissue T by advancing the endoscope. As a result, as illustrated in FIG. 16, the pair of arms 21 swing around the rotation axis RO, and the clip unit 100B assumes a swing configuration. The pair of arms 21 are disposed so that a front direction F faces the front of the tissue T, which is a treatment region.

According to the clip unit 100B of the present embodiment, the tissue T can be suitably grasped in a lumen. Even when the tissue T is treated by a tangential approach, the pair of arms 21 can be disposed to face the front of the tissue T, which is a treatment region, by swinging the entire clip unit 100B. Since the entire clip unit 100B swings, the clip unit 100B can be easily disposed to face the front of the tissue T even in a narrow lumen.

While the second embodiment of the present invention has been described in detail as above with reference to the drawings, the specific configurations are not limited to the embodiment and may include design changes or the like within a range not departing from the gist of the present invention. Also, the components illustrated in the above-described embodiment and modified examples can be configured by appropriately combining them.

### Third embodiment

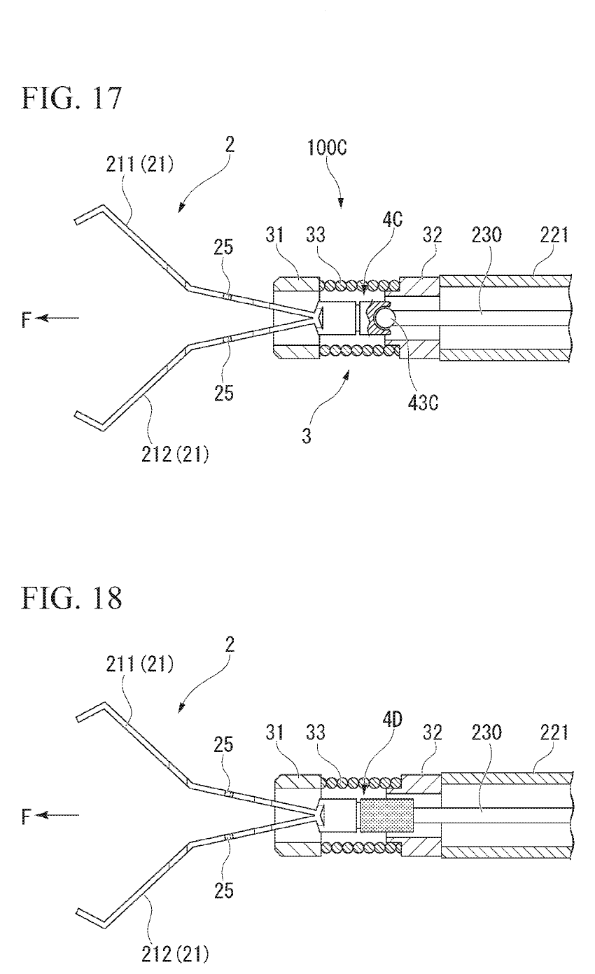

A third embodiment of the present invention will be described with reference to FIG. 17. In the following description, components that are common to those already described will be denoted by the same reference signs and duplicate description will be omitted.

FIG. 17 is a view illustrating a clip unit 100C according to the present embodiment.

The clip unit 100C is loaded into a distal end tip (distal end portion) 221 of a sheath 220 of an applicator 200, similarly to the clip unit 100 of the first embodiment.

The clip unit 100C includes a clip 2, a pressing tube (tubular member) 3, and a curved member (connection member) 4C.

The curved member (connection member) 4C is a ball joint, and supports a pair of arms 21 to be swingable (rotatable) about a ball 43C. In the present embodiment, a swinging direction (rotating direction) Q of the pair of arms 21 about the ball 43C as a rotation center is omnidirectional.

The clip unit 100C can perform both the operation of the clip unit 100 of the first embodiment and the operation of the clip unit 100B of the second embodiment.

According to the clip unit 100C of the present embodiment, the tissue T can be suitably grasped in a lumen. Even when the tissue T is treated by a tangential approach, the pair of arms 21 can be disposed to face the front of the tissue T, which is a treatment region, by swinging the entire clip unit 100C. Since the entire clip unit 100C swings, the clip unit 100C can be easily disposed to face the front of the tissue T even in a narrow lumen.

While the third embodiment of the present invention has been described in detail as above with reference to the drawings, the specific configurations are not limited to the embodiment and may include design changes or the like within a range not departing from the gist of the present invention. Also, the components illustrated in the above-described embodiment and modified examples can be configured by appropriately combining them.

### Modified example 4

FIG. 18 is a view illustrating a curved member 4D which is a modified example of the curved member 4C.

The curved member 4D is an elastic member such as rubber. A swinging direction (rotating direction) Q of the pair of arms 21 about the curved member 4D as a rotation center is omnidirectional.

### Fourth embodiment

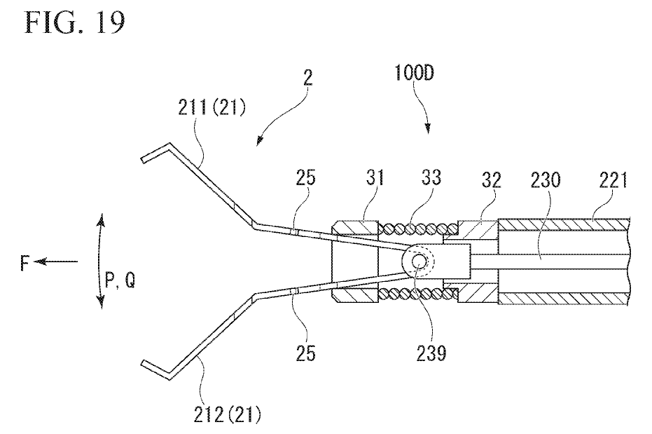

A fourth embodiment of the present invention will be described with reference to FIG. 19. In the following description, components that are common to those already described will be denoted by the same reference signs and duplicate description will be omitted.

FIG. 19 is a view illustrating a clip unit 100D according to the present embodiment.

The clip unit 100D is loaded into a distal end tip (distal end portion) 221 of a sheath 220 of an applicator 200, similarly to the clip unit 100 of the first embodiment.

The clip unit 100D includes a clip 2 and a pressing tube (tubular member) 3. The clip unit 100D does not have a curved member (connection member) 4. The clip 2 is hooked onto a hook 239 provided at a distal end of the sheath. A swinging direction (rotating direction) Q of the clip 2 substantially coincides with an opening/closing direction P of a pair of arms 21 as in the first embodiment.

The clip 2 may be connected to a ball joint provided at the distal end of the sheath. In this case, the swinging direction (rotating direction) Q of the clip 2 is omnidirectional as in the third embodiment.

According to the clip unit 100D of the present embodiment, the tissue T can be suitably grasped in a lumen. Even when the tissue T is treated by a tangential approach, the pair of arms 21 can be disposed to face the front of the tissue T, which is a treatment region, by swinging the entire clip unit 100D. Since the entire clip unit 100D swings, the clip unit 100D can be easily disposed to face the front of the tissue T even in a narrow lumen.

While the fourth embodiment of the present invention has been described in detail as above with reference to the drawings, the specific configurations are not limited to the embodiment and may include design changes or the like within a range not departing from the gist of the present invention. Also, the components illustrated in the above-described embodiment and modified examples can be configured by appropriately combining them.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A clip device comprising:
a clip unit having an arm which opens and closes;
a wire connected to the clip unit; and
a sheath through which the wire is inserted and which extends in a longitudinal axis direction, wherein
the clip unit is separably connected to a distal end part of the sheath, and
the clip unit is transitionable between:
a first configuration in which the clip unit opens and closes about a central axis of the sheath; and
a second configuration in which the clip unit opens and closes about an axis intersecting the central axis.

2. The clip device according to claim 1, wherein the clip unit is in the first configuration when it is separated from the sheath and left indwelling.

3. The clip device according to claim 1, wherein a swinging direction in which the clip unit transitions between the first configuration and the second configuration is the same as an opening/closing direction of the arm.

4. The clip device according to claim 1, wherein a swinging direction in which the clip unit transitions between the first configuration and the second configuration is orthogonal to an opening/closing direction of the arm.

5. The clip device according to claim 1, wherein
the clip unit has a tubular member which is able to accommodate at least a part of the arm, and
the tubular member has a flexible tube portion which is deformable in accordance with the transition of the clip unit when the clip unit transitions between the first configuration and the second configuration.

6. The clip device according to claim 5, wherein the flexible tube portion has elasticity to return the clip unit to the first configuration.

7. The clip device according to claim 5, wherein the clip unit has a curved member connecting the arm and the wire and causing the arm to transition between the first configuration and the second configuration.

8. The clip device according to claim 7, wherein
the tubular member has a lock mechanism which locks the arm in a closed configuration when at least a part of the arm is accommodated therein, and
a rotating portion of the curved member is disposed in an internal space of the flexible tube portion when the arm is not locked in the closed configuration.

9. A clip device, which is a reloadable clip device, comprising:
a clip unit having an arm which opens and closes;
a wire connected to the clip unit; and
a sheath through which the wire is inserted and which extends in a longitudinal axis direction, wherein
the clip unit is separably connected to a distal end part of the sheath, and
the clip unit is transitionable between:
a first configuration in which the clip unit opens and closes about a central axis of the sheath; and
a second configuration in which the clip unit opens and closes about an axis intersecting the central axis.

10. The clip device according to claim 9, wherein
the clip unit has a tubular member which is able to accommodate at least a part of the arm, and
the tubular member has a flexible tube portion which is deformable in accordance with the transition of the clip unit when the clip unit transitions between the first configuration and the second configuration.

11. The clip device according to claim 10, wherein the clip unit has a curved member connecting the arm and the wire and causing the arm to transition between the first configuration and the second configuration.

12. The clip device according to claim 11, wherein
the tubular member has a lock mechanism which locks the arm in a closed configuration when at least a part of the arm is accommodated therein, and
a rotating portion of the curved member is disposed in an internal space of the flexible tube portion when the arm is not locked in the closed configuration.

13. A clip unit, which is a clip unit loaded into a sheath of an applicator, comprising:
an arm which opens and closes; and
a tubular member which is able to accommodate at least a part of the arm, and
being able to transition between:
a first configuration in which the clip unit opens and closes about a central axis of the sheath; and
a second configuration in which the clip unit opens and closes about an axis intersecting the central axis.

14. The clip unit according to claim 13, being in the first configuration when it is separated from the sheath and left indwelling.

15. The clip unit according to claim 13, wherein a swinging direction for transitioning between the first configuration and the second configuration is the same as an opening/closing direction of the arm.

16. The clip unit according to claim 13, wherein a swinging direction for transitioning between the first configuration and the second configuration is orthogonal to an opening/closing direction of the arm.

17. The clip unit according to claim 13, wherein the tubular member has a flexible tube portion which is deformable in accordance with the transition of the clip unit when the clip unit transitions between the first configuration and the second configuration.

18. The clip unit according to claim 17, wherein the flexible tube portion has elasticity to return the clip unit to the first configuration.

19. The clip unit according to claim 17, comprising a curved member connecting the arm and a wire which is inserted through the sheath, and configured to cause the arm to transition between the first configuration and the second configuration.

20. The clip unit according to claim 19, wherein
the tubular member has a lock mechanism which locks the arm in a closed configuration when at least a part of the arm is accommodated therein, and
a rotating portion of the curved member is disposed in an internal space of the flexible tube portion when the arm is not locked in the closed configuration.
